(19) United States
(12) Patent Application Publication
(10) Pub. No.: US 2015/0250459 A1
(43) Pub. Date: Sep. 10, 2015

(54) Carbon Fiber Medical Instrument

(71) Applicant: BP II Technologies, Carlsbad, CA (US)

(72) Inventors: Douglas M. Arm, Carlsbad, CA (US); Bradford A. Conlan, Solana Beach, CA (US); Kenneth E. Carrier, Wakefield, MA (US); Hoi Sang U, Rancho Santa Fe, CA (US); Horst Fichtner, Carlsbad, CA (US); J. Peter Amis, Encinitas, CA (US); Robert K. Shanahan, Carlsbad, CA (US)

(73) Assignee: BP II Technologeis, Carlsbad, CA (US)

(21) Appl. No.: 14/197,026

(22) Filed: Mar. 4, 2014

(51) Int. Cl. A61B 17/00; A61M 5/142; A61B 18/14

(52) U.S. Cl. CPC A61B 17/00; A61B 18/1482; A61M 5/142; A61M 2205/0238; A61M 2205/0244; A61M 2205/0272; A61B 2017/00526; A61B 2018/00964

(57) ABSTRACT

A medical instrument is disclosed that includes an elongated probe section having a length (L) between a proximal and a distal probe section end. The probe section includes a plurality of carbon fibers that is arrayed along the axis and embedded in a medical grade, biocompatible epoxy resin. The probe section is constructed to be rigid by controlling the density of carbon fibers such that the maximum predetermined off-axis deflection (dₘₐₓ) is less than about 30% of the probe section length (dₘₐₓ<0.3 L). A handle is affixed, and positioned to be coaxial with, the proximal end of the probe section. For the instrument, the probe section is constructed of materials that are impervious to an imaging modality (e.g. magnetic fields). For example, with this construction, the probe section can be immersed in a homogenous magnetic field that is established by an MRI system without distorting the resulting MRI image.

### FIELD OF THE INVENTION

The present invention pertains generally to medical instruments. More particularly, the present invention pertains to medical instruments which can be used with various imaging modalities. The present invention is particularly, but not exclusively, useful as a surgical probe that can be used in an MRI magnetic field without distorting the images obtained by the MRI system.

### BACKGROUND OF THE INVENTION

Magnetic Resonant Imaging (MRI) and similar systems are widely used to produce images of the internal structures of the human body. In some cases, images can be produced and used during a surgical procedure. In the event, images obtained can be used to position surgical equipment, including probes, relative to anatomical features. In addition, MRI imaging can be used to view, in near real time, the results of surgical procedures. For example, the image can provide information regarding process steps within a surgical procedure, such as the creation of an incision, the injection of a medicament, the cauterization of tissue, and the like.

With the above in mind, some materials, when immersed in the magnetic field generated during MRI imaging, interfere with the magnetic field and can cause distortions in the resulting MRI images. For example, when a probe is constructed from one of these interfering materials, the resultant MRI images tend to visually enlarge the probe and, thus, degrade the precision of the image. In particular, non-ferrous materials such as Aluminum and Titanium, which may be considered as candidates to construct surgical probes, can interfere with a magnetic field, resulting in artifacts that cause distortions in MRI images.

In addition to a material's effect on an MRI magnetic field, other factors must be considered during the selection of materials for construction of medical probes and similar equipment. For example, the selected materials must be biocompatible. This is particularly true for materials that are introduced into the body and placed in contact with tissue and other bodily fluids. In addition, the strength and stiffness of the material must often be considered. This can be especially true when a probe having a relatively large aspect ratio (i.e. a relatively long, thin probe) is contemplated. This is often the case because it is often desirable to reach deep within a bodily organ with as small an access orifice as possible. Moreover, failure of the probe to maintain rigidity during insertion and placement within the body can result in the probe being directed off course and missing the intended treatment location. For the case where the probe is also used to deliver a fluid to a treatment area, loss of rigidity can also adversely affect the flow of fluid through the relatively small diameter probe lumen.

In some instances, a probe may be used to transfer an electrical current to a treatment site, for example, to heat, excite, stimulate or cauterize tissue. For these types of procedures, a conductive path must be maintained along the length of the probe. This, in turn implies the use of a conductive material to construct the probe.

In light of the above it is an object of the present invention to provide a medical instrument made of materials that do not interfere with an MRI magnetic field or cause distortions in the resulting MRI images. Another object of the present invention is to provide surgical probes that are conductive, made of biocompatible materials, and are sufficiently rigid to allow the probe to be placed at an intended treatment location in the body. Still another object of the present invention is to provide a medical instrument, and corresponding methods for manufacturing and using the medical instrument, that are simple to implement, easy to use and comparatively cost effective.

### SUMMARY OF THE INVENTION

In accordance with the present invention, a medical instrument that is impervious to a magnetic field includes an elongated probe section having a length (L) between a proximal probe section end and a distal probe section end. Structurally, the probe section includes a plurality of carbon fibers that is embedded in a medical grade, biocompatible epoxy resin. For the medical instrument, the probe section defines a longitudinal axis and is constructed with the carbon fibers arrayed along the axis to extend between the proximal end and the distal end of the probe section. In one embodiment, the carbon fibers in the probe section are arrayed with a substantially uniform density in a radial direction from the axis.

With the above described arrangement, the probe section is formed with sufficient structural rigidity to resist substantial off-axis deflection. In more quantitative terms, in a first embodiment, a relatively thin, substantially cylindrical probe section is contemplated having a probe section outer diameter (Dₒ) in a range between 500 and 1,500 microns. In another embodiment, the probe section is tapered with a decreasing outer diameter (Dₒ) in a direction from the proximal end to the distal end. For these embodiments, the probe section is constructed with a maximum predetermined off-axis deflection (dₘₐₓ) of the distal probe section end relative to the proximal probe section end that is less than about 30% of the probe section length (dₘₐₓ<0.3 L). For example, the probe section can be designed with a specified density of carbon fibers to provide the required structural rigidity.

Also for the medical instrument of the present invention, a handle is affixed to, and positioned to be coaxial with, the proximal end of the probe section. With this cooperative interaction of structure, the handle can be used to manipulate the probe section during a surgical procedure that is performed while the probe section is immersed within an MRI magnetic field. Specifically, for the present invention, it is envisioned that at least a portion of the probe section will be immersed in a homogenous magnetic field that is established by an MRI system to produce an MRI image.

In one embodiment of the present invention, the medical instrument includes a probe section that is formed with a lumen that extends along the probe section axis between the proximal and distal probe section ends. For this embodiment, the medical instrument can include a fluid source that is connected in fluid communication with the proximal end of the probe section. A pump is also provided that is connected with the fluid source to transfer a fluid from the fluid source and through the probe section lumen for expulsion of the fluid from the distal end of the probe section. For the embodiments where the probe section has a lumen, the inner diameter (Dᵢ) of the lumen is typically less than about 80% of the outer diameter (Dₒ) of the probe section (Dᵢ/Dₒ<0.8).

In one implementation of the present invention, the probe section can be constructed to provide a conductive pathway from the proximal probe section end to the distal probe section end. For example, this conductive pathway can extend through the carbon fibers. For this implementation, the medical instrument can include a voltage source that is connected to the distal end of the probe section by way of a switch. In use, the switch is operable to selectively send an electrical current from the voltage source through the carbon fibers in the probe section from the proximal end to the distal end of the probe section. For example, a current can be applied to internal tissue to treat the tissue, for example, by stimulation/excitation or cauterization.

The probe section of a surgical probe can be manufactured in accordance with the present invention by first orienting a plurality of carbon fibers along a linear axis. In one embodiment, the carbon fibers are oriented to establish a unidirectional composite material wherein the carbon fibers are aligned substantially parallel to the axis. In another embodiment, the carbon fibers are oriented to establish a helical pattern around the axis with each carbon fiber inclined with a positive pitch angle (+α₁) relative to the axis. Once oriented, the carbon fibers are then embedded in an epoxy resin and the embedded fibers are wrapped within a woven material made of carbon fibers to produce an assembly. Next, the assembly is processed, e.g. by applying a selected temperature/pressure regimen, to cure the epoxy resin and create an elongated probe section. The handle can then be affixed to the proximal end of the probe section.

To manufacture a probe section having a lumen, a mandrel is first aligned along a linear axis and the carbon fibers are arrayed along the mandrel. Once arrayed, the carbon fibers are embedded in epoxy resin and the embedded fibers are wrapped within a woven material made of carbon fibers to produce an assembly. The assembly is then processed, e.g. by applying a selected temperature/pressure regimen to cure the epoxy resin and create an elongated probe section. The mandrel is then removed from the probe section after the curing step to form a lumen in the probe section.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of components for a system which incorporates a medical instrument of the present invention;

FIG. 2 is a perspective view of a medical instrument in accordance with the present invention;

FIG. 3A is a perspective view of a probe section for use in a preferred embodiment of the medical instrument;

FIG. 3B is a perspective view of the probe section shown in FIG. 3A which includes a sheath;

FIG. 4A is a cross-section view of the probe section as seen along the line 4-4 in FIG. 3A;

FIG. 4B is a cross-section view of the probe section as seen along the line 4-4 in FIG. 3B;

FIG. 5A is a perspective view of a mandrel for use in the manufacture of an alternate embodiment of the medical instrument;

FIG. 5B is a perspective view of a probe section for use in an alternate embodiment of the medical instrument, shown during manufacture;

FIG. 5C is a perspective view of the probe section shown in FIG. 5B which includes a sheath;

FIG. 5D is a perspective view of the probe section shown in FIG. 5C, after removal of the mandrel to expose a lumen through the manufactured probe section;

FIG. 6A is a cross-section view of the mandrel as seen along the line 6-6 in FIG. 5A;

FIG. 6B is a cross-section view of the probe section as seen along the line 6-6 in FIG. 5B;

FIG. 6C is a cross-section view of the probe section as seen along the line 6-6 in FIG. 5C;

FIG. 6D is a cross-section view of the probe section as seen along the line 6-6 in FIG. 5D; and

FIG. 7 is a schematic presentation showing an interweaving of carbon fibers along helical paths having respectively alternating positive and negative pitch angles for a manufacture of a probe section or a sheath in accordance with the present invention.

### DESCRIPTION OF THE PREFERRED EMBODIMENTS

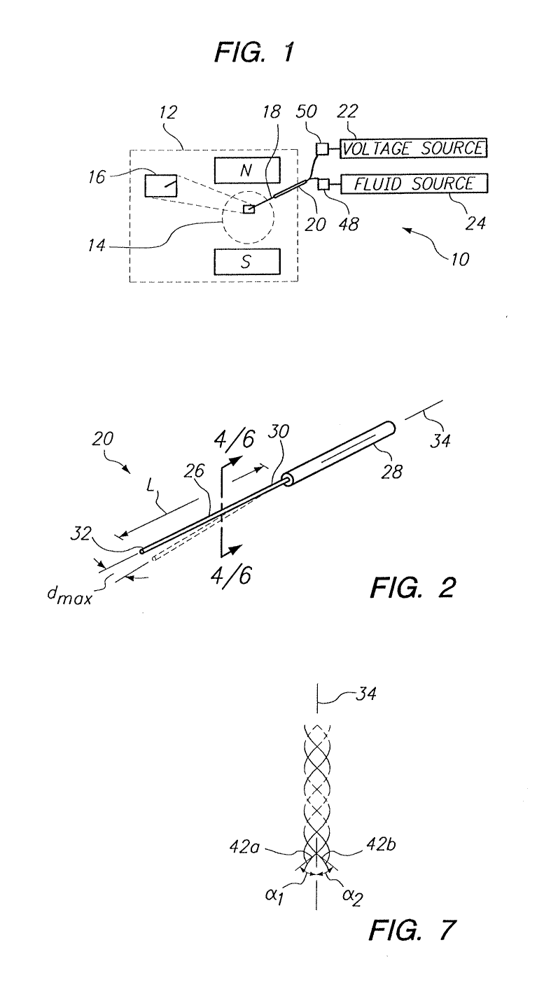

Referring initially to FIG. 1, a system for performing a surgical procedure is shown and generally designated 10. As shown, the system 10 includes an MRI sub-system 12 that establishes a homogenous magnetic field 14 and produces an MRI image 16. FIG. 1 also shows that the system 10 includes a medical instrument 18 which can include a probe assembly 20 that is operably coupled with a voltage source 22 and a fluid source 24.

FIG. 2 shows the probe assembly 20 in greater detail. As seen there, the probe assembly includes an elongated probe section 26 and a handle 28. The probe section 26 extends a length (L) along a longitudinal axis 34 from a proximal end 30 at the handle 28 to a tip at a distal end 32 of the probe section 26. For example, a typical probe section 26 may have a length (L) in the range of about 1 inch to about 20 inches. FIG. 2 also illustrates that for the present invention, the probe section 26 can be constructed with a maximum predetermined off-axis deflection (dₘₐₓ) of the distal end 32 relative to the proximal end 30 that is less than about 30% of the probe section length (dₘₐₓ<0.3 L). To achieve this structural rigidity, the probe section 26 can be designed with a pre-selected density of carbon fibers. As envisioned for the present invention, the probe section 26 may be any type instrument known in the pertinent art, such as a rod, a needle, a tube, a tweezers or an awl.

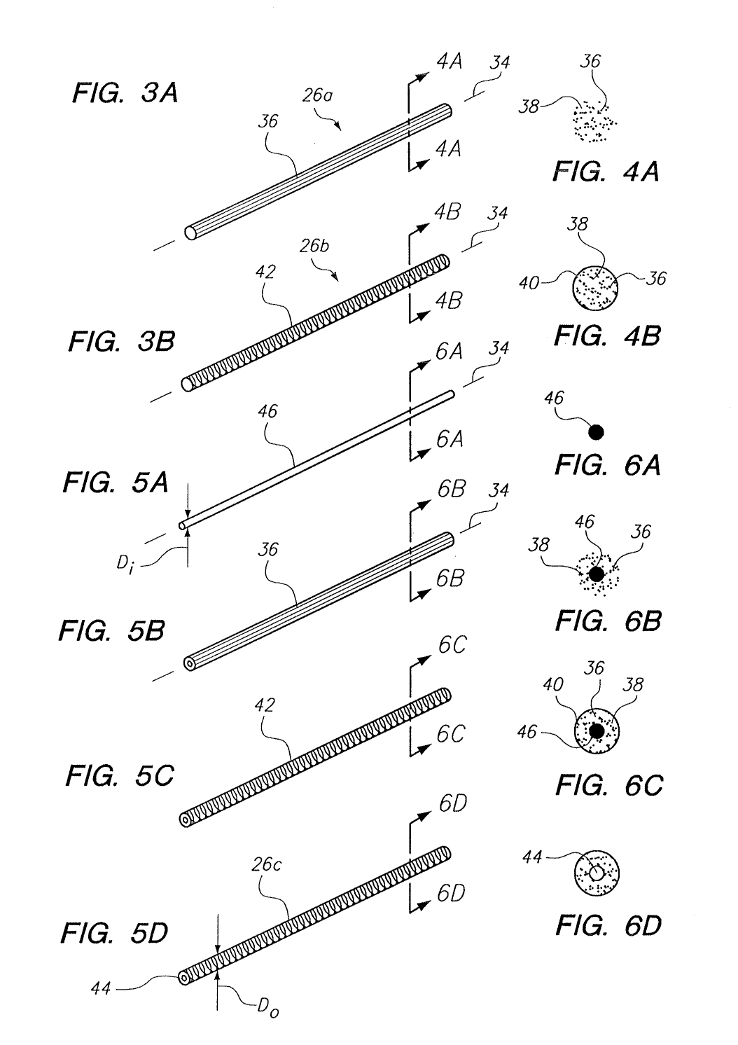

FIGS. 3A and 4A show a first embodiment of a probe section 26a having a plurality of carbon fibers 36 that is embedded in a medical grade, biocompatible epoxy resin 38. For example, the graphite fibers can be relatively thin fibers having outer diameters of about five microns, and the epoxy resin 38 can be a biocompatible, United States Pharmacopoeia class VI epoxy (USP6). For the embodiment shown in FIGS. 3A and 4A, the carbon fibers 36 are oriented to establish a unidirectional composite material wherein the carbon fibers 36 are aligned substantially parallel to the axis 34.

FIGS. 3B and 4B show another embodiment of a probe section 26b having a plurality of carbon fibers 36 that is embedded in a medical grade, biocompatible epoxy resin 38. For the embodiment shown in FIGS. 3B and 4B, internal carbon fibers 36 are oriented to establish a unidirectional composite material wherein the carbon fibers 36 are aligned substantially parallel to the axis 34. In addition, as shown, a sheath 40 surrounds the carbon fibers 36. The sheath 40 can include carbon fibers 42 that are oriented to establish a helical pattern around the axis 34. FIG. 7 shows such an arrangement in which carbon fibers 42a are inclined with a positive pitch angle (+α₁) relative to the axis 34 and carbon fibers 42b are inclined with a negative pitch angle (−α₂) relative to the axis 34. In one embodiment, the carbon fibers 42a,b are inclined with α₁=α₂, and wherein α₁ and α₂ are in a range between about 0° and about 60°.

To prepare the probe section 26b, the plurality of carbon fibers 36 are oriented along the linear axis 34 and embedded in the resin 38 to establish a unidirectional composite material. The embedded carbon fibers 36 are then wrapped within a woven material made of carbon fibers to produce the sheath 40. Next, the assembly is processed, e.g. by applying a selected temperature/pressure regimen, to cure the epoxy resin 38 and create the probe section 26b. The handle 28 (see FIG. 2) can then be affixed to the proximal end 30 of the probe section 26.

Referring back to FIG. 2, it is illustrated that for the present invention, the probe section 26 can be constructed with a maximum predetermined off-axis deflection (dₘₐₓ) of the distal end 32 relative to the proximal end 30 that is less than about 30% of the probe section length (dₘₐₓ<0.3 L). To achieve this structural rigidity, the probe section 26 can be designed with a pre-selected density of carbon fibers 36, 42 (FIGS. 3A, 3B).

FIGS. 5A-5D and corresponding FIGS. 6A-6D, illustrate, in sequence, a series of process steps to produce a tube shaped probe section 26c having a lumen 44 (shown in FIGS. 5D and 6D). Beginning with FIGS. 5A and 6A, it can be seen that the process begins by aligning a mandrel 46 along an axis 34. Next, as seen in FIGS. 5B and 6C the carbon fibers 36 are arrayed along the mandrel 46. For the embodiment shown, the plurality of carbon fibers 36 is oriented along the linear axis 34 and embedded in the resin 38 to establish a unidirectional composite material. Next, as shown in FIGS. 5C and 6C, the embedded fibers 36 are wrapped within a woven material made of carbon fibers 42 to produce a sheath 40. The assembly shown in FIGS. 5C and 6C is then processed, e.g. by applying a selected temperature/pressure regimen to cure the epoxy resin 38 and to create an elongated probe section. FIGS. 5D and 6D show the probe section 26c after the mandrel 46 (FIG. 5A) has been removed to form the lumen 44 in the probe section 26c.

For the cylindrical probe section 26a-c shown in FIGS. 3A, 3B and 5D, it is contemplated that the probe section 26a-c has a probe section outer diameter (Dₒ), as illustrated in FIG. 5D in a range between 500 and 1,500 microns. In addition, for the embodiment shown in FIG. 5D in which the probe section 26c has a lumen 44, the inner diameter (a) of the lumen 44 (diameter (a) illustrated in FIG. 5A) is typically less than about 80% of the outer diameter (Dₒ) of the probe section 26c (Dᵢ/Dₒ<0.8).

Cross-referencing FIG. 1 and FIG. 5D, for the present invention, a fluid source 24 can be connected in fluid communication with the proximal end 30 (FIG. 2) of the probe section 26c. A pump 48 is connected with the fluid source 24 to transfer a fluid (e.g. fluid medicament, therapeutics, biologics and cells) from the fluid source 24 and through the probe section lumen 44 for expulsion of the fluid from the distal end 32 (FIG. 2) of the probe section 26c. For example, this arrangement can be used to treat a localized region of target tissue by infusing the region with a fluidic medicament.

FIG. 1 also illustrates that the probe assembly 20 can be constructed to provide a conductive pathway from a voltage source 22 to a distal end 32 (FIG. 2) of a probe section 26. For example, this conductive pathway can extend through conductive carbon fibers that are located in the handle 28 and probe section 26. As shown in FIG. 1, the voltage source 22 is connected to the distal end 28 (FIG. 2) of the probe section 26 by way of a switch 50. In use, the switch 50 is operable to selectively send an electrical current from the voltage source 22 through the carbon fibers in the handle 28 and probe section 26, for example, to cauterize internal tissue.

The operation of the present invention can best be appreciated with cross-reference to FIGS. 1 and 2. As seen there, the handle 28 can be used to manipulate the probe section 26 during a surgical procedure that is performed while the probe section 26 is immersed within a homogenous magnetic field 14 that is established by an MRI sub-system 12 to produce an MRI image 16. The medical instrument 18 can be used to treat tissue in a bodily organ, such as the brain, while one or more MRI image(s) 16 are obtained to properly position the distal end 32 of the probe section 26 at the intended target and/or determine the progress of a treatment procedure. Treatments can include, but are not necessarily limited to, cauterization and/or infusion, as described above.

While the particular Carbon Fiber Medical Instrument as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An instrument impervious to an imaging modality which comprises:
an elongated probe section having a proximal end and a distal end, wherein the probe section defines a longitudinal axis and includes a plurality of carbon fibers embedded in an epoxy resin, with the carbon fibers arrayed along the axis to extend between the proximal end and the distal end of the probe section, to provide a structural rigidity therefor characterized by a predetermined maximum off-axis deflection (dₘₐₓ) of the distal end relative to the proximal end of the probe section; and
a handle affixed to the proximal end of the probe section for use in manipulating the probe section during a surgical procedure performed within the influence of the imaging modality.

2. An instrument as recited in claim 1 wherein the imaging modality is selected from the group consisting of a magnetic field, an electrical field, an electromagnetic field and an acoustic field.

3. An instrument as recited in claim 2 wherein the magnetic field is a homogeneous magnetic field and is created by a Magnetic Resonance Imaging (MRI) device.

4. An instrument as recited in claim 1 wherein the handle is coaxial with the probe section and includes a plurality of carbon fibers embedded in the epoxy resin, and wherein the probe section has a length (L) and the predetermined deflection (dₘₐₓ) is less than 30% of the length (dₘₐₓ<0.3 L).

5. An instrument as recited in claim 1 wherein the probe section is formed with a lumen extending therethrough along the axis between the proximal end and the distal end.

6. An instrument as recited in claim 5 further comprising:
a fluid source connected in fluid communication with the proximal end of the probe section; and
a pump connected with the fluid source for transferring a fluid therefrom and through the probe section for expulsion of the fluid from the distal end of the probe section.

7. An instrument as recited in claim 5 wherein the probe section is substantially cylindrical and an outer diameter (Dₒ) of the probe section is in a range between 500 and 2,500 microns and wherein an inner diameter (Dᵢ) of the lumen is less than 80% of Dₒ (Dᵢ/Dₒ<0.8).

8. An instrument as recited in claim 6 wherein the fluid is selected from the group consisting of a fluid medicament, therapeutics, biologics and cells.

9. An instrument as recited in claim 1 wherein the probe section is tapered with a decreasing outer diameter (Dₒ) in a direction from the proximal end to the distal end.

10. An instrument as recited in claim 1 wherein carbon fibers in the probe section are arrayed with a substantially uniform density in a radial direction from the axis.

11. An instrument as recited in claim 10 further comprising:
a voltage source connected to the distal end of the probe section; and
a switch for selectively sending an electrical current from the voltage source through the carbon fibers in the probe section to the distal end of the probe section.

12. A method for manufacturing an instrument impervious to an imaging modality which comprises the steps of:
orienting a plurality of carbon fibers along a linear axis;
embedding the carbon fibers in an epoxy resin;
wrapping the embedded fibers within a woven material, wherein the woven material is made of carbon fibers;
curing the epoxy resin to create an elongated probe section for the probe, wherein the probe section has a proximal end and a distal end, and the carbon fibers are arrayed along the axis to extend between the proximal end and the distal end of the probe section; and
affixing a handle to the proximal end of the probe section, wherein the handle is coaxial with the probe section and includes a plurality of carbon fibers embedded into the epoxy resin.

13. A method as recited in claim 12 further comprising the steps of:
establishing a length (L) for the probe section; and
creating a density of carbon fibers in the probe section during the embedding step to provide a structural rigidity therefor characterized by a predetermined maximum off-axis deflection (dₘₐₓ) of the distal end relative to the proximal end of the probe section, wherein the predetermined deflection (dₘₐₓ) is less than 30% of the length (dₘₐₓ<0.3 L).

14. A method as recited in claim 12 further comprising the steps of:
using a mandrel to define the linear axis, wherein the orienting step is accomplished by arraying the carbon fibers along the mandrel; and
removing the mandrel from the probe section after the curing step to form a lumen extending therethrough along the axis between the proximal end and the distal end, wherein the probe section is substantially cylindrical and an outer diameter (Dₒ) of the probe section is in a range between 500 and 1,500 microns, and wherein an inner diameter (Dᵢ) of the lumen is less than 80% of Dₒ (Dᵢ/Dₒ<0.8).

15. A method as recited in claim 12 wherein the orienting step is accomplished to establish a unidirectional composite material wherein all carbon fibers are aligned substantially parallel to the axis.

16. A method as recited in claim 12 wherein the orienting step is accomplished to establish a first plurality of carbon fibers with each carbon fiber having a helical pattern around the axis, and wherein each carbon fiber is inclined with a positive pitch angle (+α₁) relative to the axis.

17. A method as recited in claim 16 further comprising a second plurality of carbon fibers having a respective helical pattern around the axis, wherein each carbon fiber is inclined with a negative pitch angle (−α₂) relative to the axis, wherein α₁=α₂, and wherein α₁ and α₂ are in a range between 0° and 60°.

18. A method for using an instrument to interact with a target region, the method comprising the steps of:
immersing the target region in a homogenous magnetic field established by a Magnetic Resonance Imaging (MRI) sub-system;
providing an elongated probe section having a proximal end and a distal end, wherein the probe section defines a longitudinal axis and includes a plurality of carbon fibers embedded in an epoxy resin, with the carbon fibers arrayed along the axis to extend between the proximal end and the distal end of the probe section, to provide a structural rigidity therefor characterized by a predetermined maximum off-axis deflection (dₘₐₓ) of the distal end relative to the proximal end of the probe section; and
affixing a handle to the proximal end of the probe section to manipulate the probe section to a location adjacent the target region.

19. A method as recited in claim 18 wherein the probe section is formed with a lumen extending therethrough along the axis between the proximal end and the distal end and wherein the method further comprises the step of connecting a fluid source in fluid communication with the proximal end of the probe section and transferring a fluid therefrom and through the probe section to infuse the target region with fluid from the distal end of the probe section.

20. A method as recited in claim 18 further comprising the step of sending an electrical current from a voltage source through the carbon fibers in the probe section to the distal end of the probe section to cauterize tissue in the target region.
